(19) United States
(12) Patent Application Publication
(10) Pub. No.: US 2011/0085934 A1
(43) Pub. Date: Apr. 14, 2011

(54) APPARATUS AND METHOD FOR STERILIZING AND DEODORIZING

(76) Inventors: Ashok V. Joshi, Salt Lake City, UT (US); John Howard Gordon, Salt Lake City, UT (US)

(21) Appl. No.: 12/899,903

(22) Filed: Oct. 7, 2010

Related U.S. Application Data
(60) Provisional application No. 61/249,944, filed on Oct. 8, 2009.

(51) Int. Cl. A61L 2/16; B01J 19/08

(52) U.S. Cl. 422/5; 422/186.12; 422/28

(57) ABSTRACT

An apparatus for sterilizing and/or deodorizing objects is disclosed herein. In one embodiment, such an apparatus includes an ozone source to provide ozone, and a hydrogen peroxide source to provide hydrogen peroxide. A mixing element intermixes the ozone and hydrogen peroxide to produce a mixture comprising oxidizing radicals. An applicator applies the mixture to an object before the oxidizing radicals decompose. The oxidizing radicals in the mixture work to oxidize organic substances, thereby interrupting the life cycle of living organisms and/or destroying or neutralizing odors. In selected embodiments, the applicator disperses the mixture into a closed environment, such as a substantially sealed room or enclosure, to sterilize and/or deodorize objects contained therein. A corresponding method is also disclosed herein.

### RELATED APPLICATIONS

This application claims priority to U.S. Provisional Patent No. 61/249,944 filed on Oct. 8, 2009 and entitled DEVICE AND METHOD FOR STERILIZING AND DEODORIZING.

### FIELD OF THE INVENTION

This invention relates to apparatus and methods for sterilizing and/or deodorizing objects.

### BACKGROUND

There is often a need to sterilize objects which have become contaminated with micro-organisms such as bacteria, viruses, or fungi, or larger organisms such as bedbugs, fleas, or lice. Objects that are contaminated with such organisms may cause the spread of disease or illness. Objects that are prone to contamination with such organisms include food items such as fresh foods, medical instruments, kitchen tools, livestock or dairy handling tools, household items such as garments, shoes, toys, door knobs, and telephones, and bathroom items such as toothbrushes, toilet seats, faucet handles, and the like. There is also frequently a need to remove or neutralize odors on objects which have become contaminated by smoke, stale body fluids, decaying organic material, or the like.

With regards to sterilizing objects, various different techniques have been developed and utilized extensively. For example, heat is often applied to kill or inactivate pathogens, or reduce the number of pathogens to a safe level. In other cases, chemicals are applied to contaminated objects where heat may damage the objects. For example, objects may be cleaned by washing them with soaps, detergents, alcohols, solvents, or the like. Other techniques for sterilizing objects include applying radiation, such as electron beams, ultraviolet light, X-rays, or gamma rays.

Various techniques have also been developed to deodorize objects. For example, adsorbents such as zeolite, activated charcoal, or silica gel may be placed on or near objects to absorb bad odors therefrom. In other cases, receptor blockers are used to block the human scent receptors from detecting certain odors. In yet other cases, masking agents are used to overwhelm bad odors with good odors. One disadvantage of masking agents is that odors may return once the masking agents dissipate.

Notwithstanding the variety of techniques that are currently being used to sterilize and/or deodorize objects, research continues to develop new and improved techniques for sterilizing and deodorizing. For example, it would be an advance in the art to provide better (e.g., safer, faster, cheaper, more effective) techniques to kill or inactivate harmful microbial and non-microbial organisms. Further needed are techniques for effectively destroying or neutralizing undesirable odors, perhaps simultaneously with and using the same principles of operation as a sterilization technique. To that end, apparatus and methods for sterilizing and/or deodorizing objects with greater efficacy are disclosed and claimed herein.

### SUMMARY

The invention has been developed in response to the present state of the art and, in particular, in response to the problems and needs in the art that have not yet been fully solved by currently available apparatus and methods. Accordingly, the invention has been developed to provide apparatus and methods to effectively sterilize and deodorize objects. The features and advantages of the invention will become more fully apparent from the following description and appended claims, or may be learned by practice of the invention as set forth hereinafter.

Consistent with the foregoing, an apparatus for sterilizing and/or deodorizing objects is disclosed herein. In one embodiment, such an apparatus includes an ozone source to provide ozone, and a hydrogen peroxide source to provide hydrogen peroxide. A mixing element intermixes the ozone and hydrogen peroxide to produce a mixture comprising oxidizing radicals. An applicator applies the mixture to an object before the oxidizing radicals decompose. The oxidizing radicals in the mixture work to oxidize organic substances, thereby interrupting the life cycle of living organisms and/or destroying or neutralizing odors. In selected embodiments, the applicator disperses the mixture into a closed environment, such as a substantially sealed room or enclosure, to sterilize and/or deodorize objects contained therein.

A corresponding method is also disclosed and claimed herein.

### BRIEF DESCRIPTION OF THE DRAWINGS

In order that the advantages of the invention will be readily understood, a more particular description of the invention briefly described above will be rendered by reference to specific embodiments illustrated in the appended drawings. Understanding that these drawings depict only typical embodiments of the invention and are not therefore to be considered limiting of its scope, the invention will be described and explained with additional specificity and detail through use of the accompanying drawings in which:

FIG. 1 is a high-level block diagram of one embodiment of an apparatus for sterilizing and/or deodorizing an object;

FIG. 2 is a high-level block diagram showing a more specific embodiment of an apparatus for sterilizing and/or deodorizing an object;

FIG. 3 is a high-level block diagram showing another more specific embodiment of an apparatus for sterilizing and/or deodorizing an object;

FIG. 4 is a high-level block diagram showing an apparatus within a substantially closed environment, such as a room or enclosure;

FIG. 5 shows an apparatus in accordance with the invention integrated with a garment bag;

FIG. 6 shows an apparatus in accordance with the invention integrated with a box, such as a garment, toy, or shoe box; and

FIG. 7 shows an apparatus in accordance with the invention incorporated into a scrub brush.

### DETAILED DESCRIPTION OF THE INVENTION

It will be readily understood that the components of the present invention, as generally described and illustrated in the Figures herein, could be arranged and designed in a wide variety of different configurations. Thus, the following more detailed description of the embodiments of the invention, as represented in the Figures, is not intended to limit the scope of the invention, as claimed, but is merely representative of certain examples of presently contemplated embodiments in accordance with the invention. The presently described embodiments will be best understood by reference to the drawings, wherein like parts are designated by like numerals throughout.

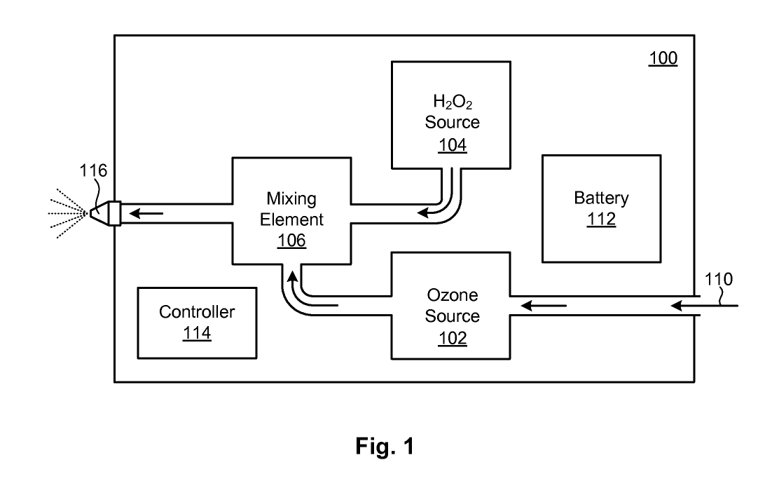

Referring to FIG. 1, a high-level block diagram of one embodiment of an apparatus 100 for sterilizing and/or deodorizing an object is illustrated. As shown, the apparatus 100 includes a source 102 of ozone (O₃) and a source 104 of hydrogen peroxide (H₂O₂). Because ozone is too unstable to obtain commercially, the ozone source 102 ideally generates the ozone at or near the time of use. Thus, in selected embodiments, the ozone source 102 is an ozone generator 102. This ozone generator 102 may require an oxygen-containing feedstock 110 (e.g., air, water, etc.) from which to generate the ozone. In one embodiment, the ozone source generates ozone from at least one of air, water, oxygen, and oxygen containing gas.

The hydrogen peroxide source 104, on the other hand, may include either a reservoir 104 containing hydrogen peroxide (which is readily obtained commercially) or a generator 104 to generate hydrogen peroxide from a feedstock such as water. A battery 112 or other internal power source 112 may provide power to the ozone source 102 and/or hydrogen peroxide source 104 as well as to other components in the apparatus 100 that require power to operate, such as the controller 114. Alternatively, an external power source may provide power to the apparatus 100 by way of an electrical cord or other means.

Upon receiving the ozone and hydrogen peroxide from the ozone source 102 and hydrogen peroxide source 104, a mixing element 106 mixes the ozone with the hydrogen peroxide. This generates an oxidizing mixture containing a high concentration of oxidizing radicals. In particular, mixing hydrogen peroxide with ozone accelerates the decomposition of the ozone which creates a higher concentration of hydroxyl radicals in the mixture. This increases the oxidation rate since hydroxyl radicals are significantly more reactive than ozone. The end result is that oxidation is more reactive and much faster using a mixture of ozone and hydrogen peroxide than using either ozone or hydrogen peroxide by themselves. This makes the mixture substantially more effective at breaking down or inactivating organic materials, such as pathogens and odor compounds. One benefit of using hydroxyl and ozone radicals is that the radicals decompose to water and oxygen, or to water and carbon dioxide when are interacting with organic molecules.

The oxidation potential of the hydroxyl radical and ozone are as follows:

| Oxidant | Formula | Voltage |
|---|---|---|
| Hydroxyl radical | OH• | 2.8 V |
| Ozone | O₃ | 2.1 V |
| Hydrogen Peroxide | H₂O₂ | 1.8 V |

As evident above, because hydroxyl radicals have an oxidation potential that is much higher than ozone, oxidation with hydroxyl radicals is much more effective than direct oxidation with ozone. The higher oxidation potential makes hydroxyl radicals more effective at breaking down organics that are difficult to oxidize such as taste and odor compounds and chlorinated organics (e.g., geosmin, MIB, phenolic compounds, trichloroethylene (TCE), and perchloroethylene (PCE)).

Upon receiving the mixture of ozone and hydrogen peroxide from the mixing element 106, an applicator 116 applies the mixture to one or more objects that are to be sterilized and/or deodorized. As mentioned above, such objects may include but are not limited to food items such as fresh foods, medical instruments, kitchen tools, livestock or dairy handling tools, household items such as garments, shoes, toys, door knobs, or telephones, and bathroom items such as toothbrushes, toilet seats, or faucet handles. In certain embodiments, the applicator 116 is a nozzle to disperse the mixture at high pressure in the form of a spray, vapor, or mist, or a piezo-electric transducer to mechanically excite the mixture to create a “fog.” This spray or fog may envelope or cover an object to provide desired sterilization and/or deodorization. In other embodiments, the applicator 116 is simply an outlet that discharges a liquid mixture. This liquid mixture may be used to sterilize and/or deodorize an object by showering or submersing the object.

In selected embodiments, a controller 114 may be provided to control the operation of the apparatus 100. For example, in selected embodiments, the controller 114 may be programmed to control the timing of operation of the apparatus 100. For example, the controller 114 may be programmed to operate the apparatus 100 during off-hours (e.g., when no people are present) or at other desirable times or intervals. In other embodiments, the controller 114 may control the flow rate through the applicator 116 using a duty cycle or by adjusting other parameters within the apparatus 100. The controller 114 may also identify when the battery 112 or other power source 112 is low or when a supply of constituents (e.g., hydrogen peroxide) is exhausted or low. The controller 114 may be programmed or controlled by a user using buttons, knobs, or other input elements known in the art, as well as provide information to a user using an output device such as a visual display, speaker, or the like.

In selected embodiments, the apparatus 100 may be configured to operate in ozone-only mode, hydrogen-peroxide-only mode, or a mode that disperses a mixture of both ozone and hydrogen peroxide. This may be accomplished using valves or other mechanisms to turn the flow of ozone and/or hydrogen peroxide on and off when needed. This may enable the apparatus 100 to operate with reduced oxidizing capability when operated in ozone-only or hydrogen-peroxide-only mode. In other embodiments, a reservoir 104 normally containing hydrogen peroxide may be filled water instead of hydrogen peroxide to reduce the oxidizing capability of the apparatus 100.

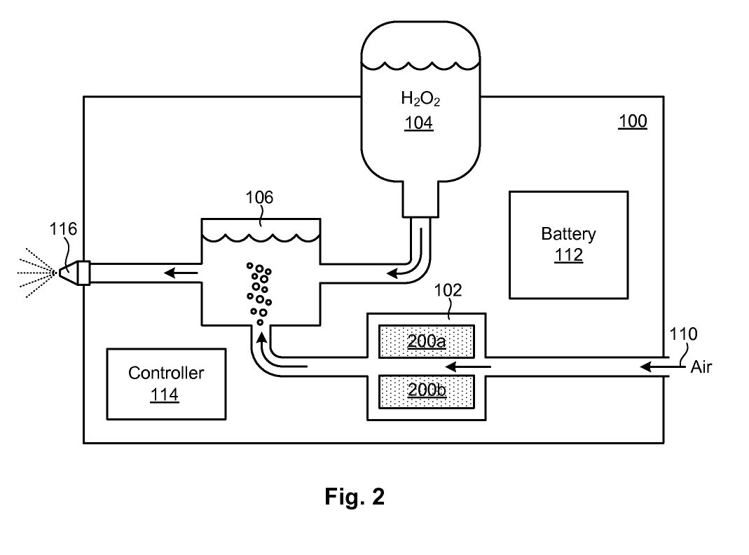

Referring to FIG. 2, a more specific embodiment of an apparatus 100 for sterilizing and/or deodorizing an object is illustrated. In this embodiment, the apparatus 100 includes a reservoir 104, such as a bottle 104, containing hydrogen peroxide. When empty, this reservoir 104 may be refilled or replaced in certain embodiments to replenish the supply of hydrogen peroxide. An ozone generator 102 may be provided to generate ozone from the oxygen contained in air 110. In this embodiment, the ozone generator 102 includes a corona generator. In certain embodiments, the corona generator generates a corona discharge between a pair of electrodes 200a, 200b using a high frequency alternating current. The corona may be used to ionize the oxygen in an oxygen-containing gas 110 (in this example air) to form ozone gas. A battery 112 or other power source may supply power to the ozone generator 102.

Once generated, the ozone gas may be passed to the mixing element 106 (along with the other residual gases in air) where it may be mixed with hydrogen peroxide. In the illustrated embodiment, the ozone gas is bubbled through the hydrogen peroxide using a bubbler 106. This will entrain the ozone in the hydrogen peroxide to produce the desired oxidizing mixture. The resulting mixture may then be applied to one or more objects using an applicator 116. In this embodiment, the applicator 116 is a nozzle 116 that generates a fine mist or spray.

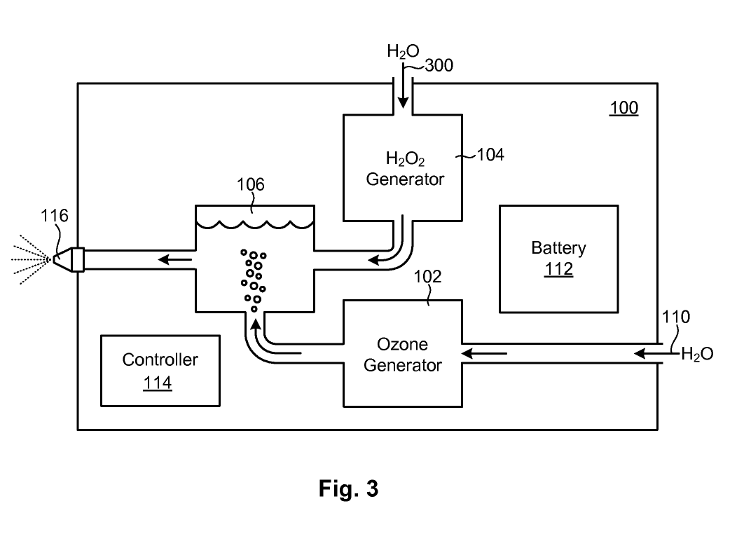

Referring to FIG. 3, another specific embodiment of an apparatus 100 for sterilizing and/or deodorizing an object is illustrated. In this embodiment, the apparatus 100 includes a hydrogen peroxide generator 104 that electrochemically generates hydrogen peroxide from water 300. One example of such a hydrogen peroxide generator 104 is disclosed in the article entitled “Improved Electrolytic Hydrogen Peroxide Generator” that was published in NASA Tech Briefs on July 2005. The article discloses a hydrogen peroxide generator which includes an electrochemical cell having electrodes located on opposite sides of a commercially available polymeric membrane. The polymeric membrane separates two half-cells. One of the half-cells produces aqueous H₂O₂.

The apparatus 100 also includes an ozone generator 102. In this embodiment, the ozone generator 102 generates ozone from the electrolytic decomposition of water 110. To accomplish this, a direct current is passed through a water solution at an anode which has a high overpotential for oxygen oxidation or evolution. Hydrogen is evolved at a cathode and, instead of having oxygen evolved at the anode, a portion of the gas evolved is ozone. Like the previous embodiment, the resulting ozone gas is bubbled through hydrogen peroxide in a bubbler 106 and the resulting mixture is applied to one or more objects by way of a nozzle 116 that generates a fine mist or spray. One of the advantages of the apparatus 100 illustrated in FIG. 3 is that the hydrogen peroxide generator 104 and ozone generator 102 both use water as a feedstock. In selected embodiments, both may draw from a common water source.

It should be recognized that the hydrogen peroxide generators 104 and ozone generators 102 described herein are simply provided by way of example and not limitation. Other devices known to generate hydrogen peroxide and/or ozone may also potentially be used in the apparatus 100. For example, it may be possible to generate ozone using ultraviolet light and/or cold plasma, as is known in the art. Thus, ozone generators 102 and hydrogen peroxide generators 104 using other principles of operation may potentially be used and are intended to be encompassed within the scope of the invention.

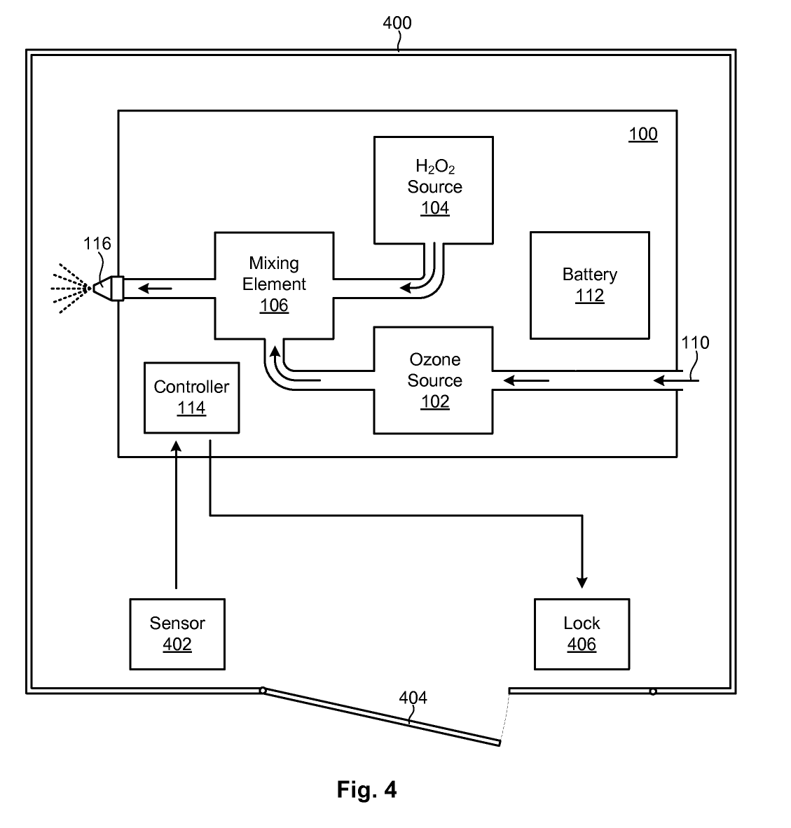

Referring to FIG. 4, in selected embodiments, the apparatus 100 may be configured to disperse the oxidizing mixture into a closed environment 400, such as a substantially closed room 400 or enclosure 400 void of humans or animals. This will ensure that the oxidizing mixture does not come into contact with humans or animals where it can cause irritation or damage to tissue. In selected embodiments, the apparatus 100 may be configured such that it functions only when it detects that a room 400 or enclosure 400 is substantially sealed. To accomplish this, the controller 114 may communicate with sensors 402 that detect when doors 404 or other barriers 404 are in open or closed positions. Once all doors 404 or barriers 404 are in closed positions, the apparatus 100 may disperse the oxidizing mixture into the room 400 or enclosure 400.

In other embodiments, the apparatus 100 may also be configured to prevent doors 404 or barriers 404 from opening until the oxidizing elements within the mixture have decomposed and are no longer a threat to humans or animals. This may be accomplished by waiting a designated time after the oxidizing mixture has been dispersed, or using sensors to detect when the level of oxidizing elements in the room 400 or enclosure 400 has reached a safe level. In certain embodiments, the controller 114 may be configured to control locks 406 on doors 404 or barriers 404 to control when humans or animals can enter the room 400 or enclosure 400, or when gases or vapors in the room 400 or enclosure 400 are safe to be released into the external environment. In certain embodiments, the controller 114 may be configured to control fans or vents to evacuate oxidizing elements from the room 400 or enclosure 400 before the room 400 or enclosure 400 is unlocked.

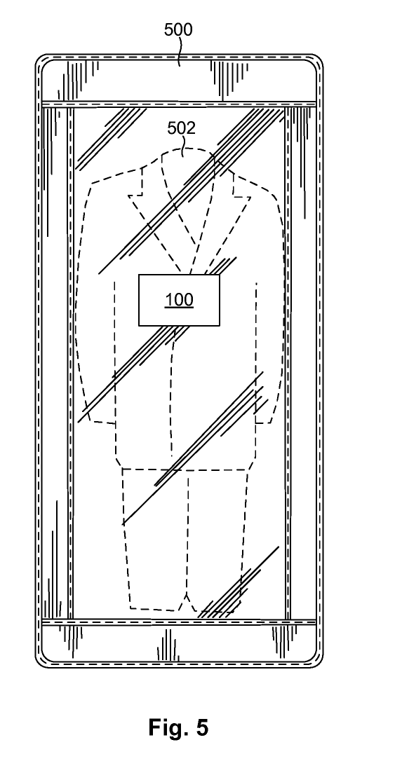

The apparatus 100 may be suitable for use with a wide variety of different rooms 400 or enclosures 400 to sterilize and/or deodorize objects contained therein. For example, the apparatus 100 may be used to dispense the oxidizing mixture into a room 400 such as an operating room, laboratory, kitchen, bathroom, food-processing room, or factory, or into an enclosure 400 such as a shoe box, closet, storage cabinet, suitcase, toy box, gym bag, garment bag, or food storage pantry. The apparatus 100 may also be integrated into devices such as scrub brushes, sprayers, sponges, mops, or other devices known in the art. Several non-limiting examples of applications for the apparatus 100 are disclosed in FIGS. 5 through 7.

Referring to FIG. 5, one embodiment of an application for an apparatus 100 in accordance with the invention is illustrated. In this embodiment, the apparatus 100 is incorporated into a garment bag 500. As shown, the apparatus 100 is attached to an outer surface of the garment bag 500 to disperse the oxidizing mixture through an opening in the garment bag 500. Alternatively, the apparatus 100 could be placed entirely inside the garment bag 500 to disperse the oxidizing mixture therein.

In operation, a user may place a garment 502 inside the garment bag 500 and seal it, such as using a zipper, velcro strip, or the like. The user may then turn the apparatus 100 on to allow it to disperse the oxidizing mixture into the bag 500. The apparatus 100 may be turned on and off manually or a timer may be used to operate the apparatus 100 over a desired time period. Once the apparatus 100 has finished dispersing the oxidizing mixture into the bag 500, a designated time period may be allowed to pass prior to opening the garment bag 500. This may give oxidizing elements in the bag 500 time to decompose. Once this time period has passed, the garment 502 may be removed from the garment bag 500 and utilized in the conventional manner.

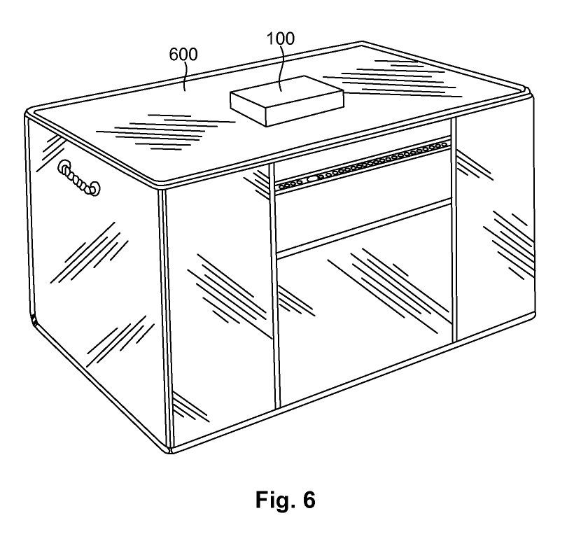

Referring to FIG. 6, one embodiment of an apparatus 100 incorporated into a box 600 such as a garment, toy, or shoe box is illustrated. As shown, the apparatus 100 is attached to an outer surface of the box 600 and is configured to disperse the oxidizing mixture through an opening in the box 600. Alternatively, the apparatus 100 may be placed inside the box 600 to disperse the oxidizing mixture therein. The oxidizing mixture envelopes or covers objects in the box 600 to sterilize and/or deodorize the objects. The oxidizing mixture may also sterilize and/or deodorize the inside surface of the box 600, thereby preventing the spread of microbes or odors to other objects that may be placed inside the box 600.

In operation, a user may place various objects inside the box 600, such as toys, garments, shoes, or the like. The user may then seal the box 600, such as by closing a zipper on the box 600, placing a lid on the box 600, or closing a door or flap on the box 600. The user may then turn the apparatus 100 on to allow it to disperse the oxidizing mixture into the box 600 and surround or envelope the objects inside the box 600. The apparatus 100 may be operated manually or be programmed to operate in an intended manner. For example, the apparatus 100 may be scheduled to operate over a desired time period. Once the apparatus 100 has completed dispersing the oxidizing mixture into the box 600, a period of time may be allowed to pass to allow the oxidizing elements to decompose. Once this time period has passed, the box 600 may be opened and the objects may be removed from the box 600 and utilized in the conventional manner.

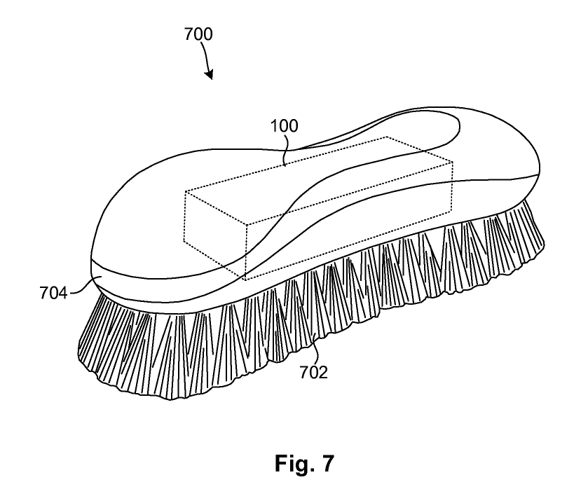

Referring to FIG. 7, one embodiment of an apparatus 100 incorporated into a scrub brush 700 is illustrated. As shown, the apparatus 100 is incorporated into a body of the scrub brush 700, although it could also be attached to an outside surface of the brush 700. The oxidizing mixture generated by the apparatus 100 is dispersed from the scrub brush 700, such as from an underside of the scrub brush 700 such that it migrates through the bristles 702, or from a front 704 of the scrub brush 700. The oxidizing mixture helps to sterilize and/or deodorize an object as it is cleaned with the scrub brush 700. In selected embodiments, the apparatus 100 includes a switch to turn on the apparatus 100 when the scrub brush 700 is being used. Alternatively, the apparatus 100 may automatically turn on when the scrub brush 700 is used. For example, a motion sensor could be used to detect movement of the scrub brush 700 to automatically activate the apparatus 100.

The present invention may be embodied in other specific forms without departing from its basic principles or essential characteristics. The described embodiments are to be considered in all respects as illustrative and not restrictive. The scope of the invention is, therefore, indicated by the appended claims rather than by the foregoing description. All changes which come within the meaning and range of equivalency of the claims are to be embraced within their scope.

1. An apparatus for sterilizing and/or deodorizing an object, the apparatus comprising:
an ozone source to provide ozone;
a hydrogen peroxide source to provide hydrogen peroxide;
a mixing element to intermix the ozone and hydrogen peroxide to produce a mixture containing oxidizing radicals; and
an applicator to apply the mixture to an object prior to decomposition of the oxidizing radicals.

2. The apparatus of claim 1, further comprising a controller to control the timing of applying the mixture.

3. The apparatus of claim 1, wherein the hydrogen peroxide source comprises at least one of a hydrogen peroxide generator and a reservoir storing hydrogen peroxide.

4. The apparatus of claim 1, wherein the ozone source comprises at least one of a corona generator and an electrolyzer.

5. The apparatus of claim 1, wherein the ozone source generates ozone from at least one of air, water, oxygen, and oxygen containing gas.

6. The apparatus of claim 1, wherein the applicator disperses the mixture into a closed environment.

7. The apparatus of claim 6, wherein the applicator comprises an atomizer to reduce the mixture to fine droplets.

8. The apparatus of claim 6, wherein the closed environment comprises at least one of a closed room and a closed enclosure.

9. The apparatus of claim 1, wherein the mixing element comprises a bubbler configured to bubble the ozone through the hydrogen peroxide.

10. The apparatus of claim 1, wherein the applicator at least one of washes the object with the mixture, and sprays the object with the mixture.

11. A method for sterilizing and/or deodorizing an object, the method comprising:
providing ozone;
providing hydrogen peroxide;
intermixing the ozone and hydrogen peroxide to produce a mixture containing oxidizing radicals; and
applying the mixture to an object before the oxidizing radicals decompose.

12. The method of claim 11, further comprising using a timer to control the timing of applying the mixture.

13. The method of claim 11, wherein providing hydrogen peroxide comprises at least one of generating hydrogen peroxide and obtaining hydrogen peroxide from a reservoir.

14. The method of claim 11, where providing ozone comprises generating ozone using at least one of a corona generator and an electrolyzer.

15. The method of claim 11, where providing ozone comprises generating ozone from at least one of air, water, oxygen, and oxygen containing gas.

16. The method of claim 11, wherein applying the mixture comprises dispersing the mixture into a closed environment.

17. The method of claim 16, wherein applying the mixture comprises atomizing the mixture.

18. The method of claim 16, wherein the closed environment comprises at least one of a closed room and a closed enclosure.

19. The method of claim 11, wherein intermixing the ozone and hydrogen peroxide comprises bubbling the ozone through the hydrogen peroxide.

20. The method of claim 11, wherein applying the mixture comprises at least one of washing the object with the mixture and spraying the object with the mixture.
